(19) United States
(12) Patent Application Publication
(10) Pub. No.: US 2026/0000610 A1
(43) Pub. Date: Jan. 1, 2026

(54) NANODISC COMPRISING MEMBRANE SCAFFOLD PROTEIN HAVING FC REGION OF ANTIBODY FUSED THERETO

(71) Applicant: MVRIX Co., Ltd., Hwaseong-si (KR)

(72) Inventors: Dae Hyuk KWEON, Seoul (KR); Jae Hyeon HWANG, Suwon-si (KR); Kyeong Won KIM, Suwon-si (KR); Jeong Hyeon YOON, Seoul (KR); Seok Oh MOON, Seoul (KR); Soo Min KIM, Anyang-si (KR)

(73) Assignee: MVRIX Co., Ltd., Hwaseong-si (KR)

(21) Appl. No.: 18/881,554

(22) PCT Filed: Jul. 5, 2023
(86) PCT No.: PCT/KR2023/009513

§ 371 Date: Feb. 13, 2025

(30) Foreign Application Priority Data
Jul. 6, 2022 (KR) 10-2022-0083203
Jun. 29, 2023 (KR) 10-2023-0084465

(51) Int. Cl. A61K 9/1274; A61K 38/48; A61K 47/42; A61P 31/12; A61P 31/16

(52) U.S. Cl. CPC A61K 9/1274; A61K 38/4813; A61K 47/42; A61P 31/12; A61P 31/16; C12Y 304/17023

(57) ABSTRACT

The present invention relates to a nanodisc comprising a membrane scaffold protein to which the Fc region of an antibody is fused. In the present invention, the Fc region of the antibody is fused to the membrane scaffold protein to improve antiviral efficacy and production yield, and thus the nanodisc prepared as described above has excellent pharmacokinetic properties.

### CROSS REFERENCE TO RELATED APPLICATIONS

This application is a National Stage of International Application No. PCT/KR2023/009513 filed Jul. 5, 2023, claiming priorities based on Korean Patent Application No. 10-2022-0083203 filed Jul. 6, 2022 and on Korean Patent Application No. 10-2023-0084465 filed Jun. 29, 2023, the entire disclosures of which are incorporated herein by reference.

### INCORPORATION BY REFERENCE OF SEQUENCE LISTING

The content of the electronically submitted sequence listing, file name: Q305434 Sequence Listing as filed; size: 16, 124 bytes; and date of creation: Dec. 31, 2024, filed herewith, is incorporated herein by reference in its entirety.

### Technical Field

The present invention relates to a nanodisc containing a membrane scaffold protein to which an Fc region of an antibody is fused and the antiviral use thereof.

### Background Art

Influenza virus is an RNA virus that belongs to the Orthomyxoviridae family and is divided into three serotypes: influenza A, influenza B, and influenza C. Thereamong, influenza B and C viruses have been confirmed to infect only humans, while influenza A virus has been confirmed to infect humans, horses, pigs, other mammals, and various types of poultry and wild birds. The serotypes of influenza A virus are classified depending on the types of two proteins, namely, hemagglutinin (HA) and neuraminidase (NA), on the surface of the virus. There are 144 types (16 types of HA proteins and 9 types of NA proteins) known to date. HA functions to attach the virus to somatic cells, and NA functions to allow the virus to penetrate into cells.

Drugs for treating viral infection that has been developed to date include M2 ion channel inhibitors such as amantadine and rimantadine, and neuraminidase inhibitors such as oseltamivir (Tamiflu®) or zanamivir (Relenza®), but these drugs have a drawback of limited effectiveness. In other words, it is known that mutant viruses resistant to amantadine- or rimantadine-based derivative compounds are quickly formed, the H5N1 type influenza viruses detected in some regions are resistant to amantadine or rimantadine-based compounds, and influenza B virus is insensitive to amantadine-based derivatives. In addition, it is known that the number of resistant viruses against oseltamivir or zanamivir-based derivative compounds is increasing and such resistant viruses frequently occur in children.

Meanwhile, many epidemics of diseases caused by viruses have occurred in recent years, such as COVID-19 caused by a coronavirus that has been ongoing since 2020.

Coronavirus is an RNA virus that belongs to the Coronavirinae subfamily of the Coronaviridae family and causes respiratory and digestive system infections in humans and animals. Coronavirus is mainly contracted through mucous membrane infection and droplet transmission, and generally causes mild respiratory infections in humans, but may also cause fatal infections, and may also cause diarrhea in cattle and pigs, and respiratory disease in chickens. Coronavirus is a representative virus that causes fatal infectious diseases in modern civilization. In April 2003, severe acute respiratory syndrome, also known as SARS, having originated in the People's Republic of China spread, and many people died, with a mortality rate of 9.6%. In 2015, Middle East Respiratory Syndrome, also known as MERS, spread from the Middle East to the world, resulting in many deaths with a mortality rate of about 36%. In addition, as the new coronaviral infection (COVID-19) originating from Wuhan, China has been confirmed around the world since December 2019, the number of infected people has been increasing.

Antibody treatments are being developed to treat viral diseases. However, antibody treatments for viral diseases are vulnerable to resistant viruses due to mutations. In addition, antiviral antibody treatments often do not have excellent therapeutic efficacy because antibodies exhibit antiviral activity by inhibiting the function of viral proteins and mediating the death of viruses by immune cells. In addition, antibody treatments may provide immunogenicity to the hosts, which increases the possibility of resistant viruses.

Specifically, virus-specific neutralizing antibody treatments (such as Regkirona and Rezenkov) were developed during the development of coronavirus treatments. Antibody treatments for coronavirus were developed quickly, but did not exhibit sufficient therapeutic effects. Dozens of coronavirus antibody treatments have been developed worldwide, but almost all antibodies including Regkirona from Celltrion, were ineffective against Omicron mutations and thus supply thereof was suspended or emergency approval was revoked. Even antibody treatment (Sotrovimab) from GSK, which was the only one effective against Omicron, was reported to trigger development resistant viruses immediately after administration.

Meanwhile, a nanodisc (ND) has a structure in which a phospholipid bilayer is surrounded in the form of a disc with membrane scaffold protein (MSP), which is a protein derived from apolipoprotein A1 (Apo-A1), which is a major ingredient of high-density lipoproteins (HDL) in the body. The nanodisc is mainly used to study the structure of various cell membrane proteins. In addition, the nanodisc (ND) acts as a carrier that delivers various physiological functional substances into the body. Advantageously, the nanodisc is a bio-derived substance and thus is stable in the body, and does not cause harmful reactions and is thus safe.

### DISCLOSURE

### Technical Problem

Therefore, the present invention has been made in view of the above problems, and it is one object of the present invention to provide a nanodisc that has excellent production yield and excellent pharmacodynamics, and is suitable for use as a carrier for delivering various physiologically functional substances into a living body.

It is another object of the present invention to provide a pharmaceutical composition for preventing or treating viral infections that exhibits excellent antiviral efficacy.

### Technical Solution

In accordance with an aspect of the present invention, the above and other objects can be accomplished by the provision of a nanodisc including a lipid bilayer being derived from phospholipid, having a flat disc-shaped bilayer structure, and including a hydrophilic group oriented outward and a hydrophobic group oriented inward, and a membrane scaffold protein (MSP) surrounding a side surface of the lipid bilayer where the hydrophobic group is exposed outside, wherein an Fc fragment is fused to the membrane scaffold protein (MSP) and protrudes outward from a side surface of the nanodisc.

The phospholipid may include at least one selected from the group consisting of phosphatidylcholine, phosphatidylserine, phosphatidylethanolamine, phosphatidylglycerol and phosphatidylinositol. Preferably, the phospholipid may include at least one selected from POPC (1-palmitoyl-2-oleoyl-sn-glycero-3-phosphocholine), DOPS (1,2-dioleoyl-sn-glycero-3-phospho-L-serine), and POPE (1-palmitoyl-2-oleoyl-sn-glycero-3-phosphoethanolamine).

The membrane scaffold protein (MSP) may be an amphipathic protein having a helix structure. The membrane scaffold protein may be apolipoprotein or a fraction of apolipoprotein that maintains the helix structure and amphipathic characteristics of the apolipoprotein.

In accordance with another aspect of the present invention, there is provided a nanodisc including a lipid bilayer being derived from phospholipid, having a flat disc-shaped bilayer structure, and including a hydrophilic group oriented outward and a hydrophobic group oriented inward, a membrane scaffold protein (MSP) surrounding a side surface of the lipid bilayer where the hydrophobic group is exposed outside, and a virus receptor hydrophobically bonded to the lipid bilayer, wherein an Fc fragment is fused to the membrane scaffold protein (MSP) and protrudes outward from the side surface of the nanodisc.

The phospholipid may include at least one selected from the group consisting of phosphatidylcholine, phosphatidylserine, phosphatidylethanolamine, phosphatidylglycerol and phosphatidylinositol.

The phospholipid may include at least one selected from the group consisting of phosphatidylcholine, phosphatidylserine, phosphatidylethanolamine, phosphatidylglycerol and phosphatidylinositol. Preferably, the phospholipid may include at least one selected from POPC (1-palmitoyl-2-oleoyl-sn-glycero-3-phosphocholine), DOPS (1,2-dioleoyl-sn-glycero-3-phospho-L-serine), and POPE (1-palmitoyl-2-oleoyl-sn-glycero-3-phosphoethanolamine).

The membrane scaffold protein (MSP) may be an amphipathic protein having a helix structure. The membrane scaffold protein may be apolipoprotein or a fraction of apolipoprotein that maintains the helix structure and amphipathic characteristics of the apolipoprotein.

The virus receptor may be angiotensin converting enzyme 2 or a compound having an end bound to sialic acid.

In accordance with another aspect of the present invention, there is provided a pharmaceutical composition for preventing or treating viral infection including a nanodisc including a lipid bilayer being derived from phospholipid, having a flat disc-shaped bilayer structure, and including a hydrophilic group oriented outward and a hydrophobic group oriented inward, a membrane scaffold protein (MSP) surrounding a side surface of the lipid bilayer where the hydrophobic group is exposed outside, and a virus receptor hydrophobically bonded to the lipid bilayer, wherein an Fc fragment is fused to the membrane scaffold protein (MSP) and protrudes outward from the side surface of the nanodisc.

The phospholipid may include at least one selected from the group consisting of phosphatidylcholine, phosphatidylserine, phosphatidylethanolamine, phosphatidylglycerol and phosphatidylinositol.

The phospholipid may include at least one selected from the group consisting of phosphatidylcholine, phosphatidylethanolamine, phosphatidylserine, phosphatidylglycerol and phosphatidylinositol. Preferably, the phospholipid may include at least one selected from POPC (1-palmitoyl-2-oleoyl-sn-glycero-3-phosphocholine), DOPS (1,2-dioleoyl-sn-glycero-3-phospho-L-serine), and POPE (1-palmitoyl-2-oleoyl-sn-glycero-3-phosphoethanolamine).

The membrane scaffold protein (MSP) may be an amphipathic protein having a helix structure. The membrane scaffold protein may be apolipoprotein or a fraction of apolipoprotein that maintains the helix structure and amphipathic characteristics of the apolipoprotein.

The virus receptor may be angiotensin converting enzyme 2 or a compound having an end bound to sialic acid.

The virus may include at least one virus selected from Coronaviridae, Bunyaviridae, Filoviridae, Flaviviridae, Hepadnaviridae, Herpesviridae, Orthomyxoviridae, Poxviridae, Rhabdoviridae, Retroviridae and Togaviridae.

### Advantageous Effects

The nanodisc of the present invention includes a membrane scaffold protein fused with the Fc region of an antibody, and has a high production yield and excellent pharmacodynamics compared to nanodiscs including general membrane scaffold proteins. In addition, when the nanodisc of the present invention further includes a virus receptor, it exhibits excellent antiviral efficacy.

### DESCRIPTION OF DRAWINGS

FIG. 1 is a schematic diagram illustrating a process of producing a nanodisc (immunodisc) according to the present invention.

FIG. 2 shows the molecular weight measured by electrophoresis after producing the membrane scaffold protein (MSP-Fc) to which the Fc region of the antibody is fused, in order to determine whether or not the membrane scaffold protein (MSP-Fc) to which the Fc region of the antibody is fused was produced intact.

FIG. 3 shows the particle size measured by dynamic light scattering (DLS) after producing the nanodisc (immunodisc) of the present invention (A of FIG. 3) and the molecular weight measured by a multi-angle light scattering (SEC-MALS) detector (B of FIG. 3) in order to determine whether or not the nanodisc (immunodisc) of the present invention was produced intact.

FIG. 4 schematically shows the differences between a nanodisc (ND) and a large nanodisc (P2N2-ND) including E. coli-expressed MSP; a nanodisc (hMSP-ND) and a large nanodisc (hMSP2N2-ND) including human-derived cells (HEK293)-expressed MSP; and a nanodisc (immunodisc) and a large nanodisc (P2N2-immunodisc) including human-derived cells (HEK293)-expressed MSP-Fc.

FIG. 5. shows the results of size exclusion FIG. 5 chromatography (SEC) using a column (Superose 6, Supedex 200) after producing the nanodisc (immunodisc, P2N2-immunodisc) of the present invention and comparison of the purification yield with that of the general nanodisc (C of FIG. 5).

FIG. 6 shows the results of size exclusion chromatography (SEC) for purification of large nanodiscs (P2N2-immunodisc-T3, T6, GD1a, 6slst, 3slst) including polysaccharide or glycolipid-based viral receptors (T3, T6, GD1a, 6slst, 3slst).

FIG. 7 shows the results of size exclusion chromatography (SEC) to determine the production yield of large nanodisc (P2N2-immunodisc-T3, T6, GD1a, 6slst, 3slst) including polysaccharide or glycolipid-based viral receptors (T3, T6, GD1a, 6slst, 3slst) (A of FIG. 7) and comparison in the purification yield with general nanodisc (B of FIG. 7).

FIG. 8 shows the results of a process of producing the nanodisc (ACE2-immunodisc) of the present invention including angiotensin converting enzyme 2 (ACE2) as a virus receptor (A of FIG. 8), a purification process using size exclusion chromatography (SEC) (B of FIG. 8), and SDS-PAGE analysis (C of FIG. 8) and dynamic light scattering (DLS) measurement (D of FIG. 8) to determine whether or not the nanodisc was produced completely.

FIG. 9 shows the results of size exclusion chromatography (SEC) to determine the excellent production yield of the nanodisc (ACE2-immunodisc) of the present invention containing angiotensin converting enzyme 2 (ACE2) as a virus receptor and comparison in purification yield with the general nanodisc.

FIGS. 10A-10C show the results of the influenza virus neutralization experiment (FIG. 10A), the influenza virus plasmid reduction experiment (FIG. 10B), and the neutralization experiment various influenza viruses

(FIG. 10C) to determine the antiviral efficacy of the nanodisc including polysaccharide or glycolipid-based viral receptors (immunodisc-GD1a, P2N2-immunodisc-T3, T6, GD1a, 6slst, 3slst).

FIG. 11 shows the results of CPE inhibition assay performed and comparison with the general nanodisc to confirm the antiviral efficacy of the nanodisc (ACE2-immunodisc) of the present invention containing angiotensin converting enzyme 2 (ACE2) as a virus receptor. Meanwhile, Vero E6 cells are cells that may be used to confirm the endocytic pathway infection among the infection pathways of coronavirus, and Calu-3 cells are cells that may be used to determine the direct fusion infection among the infection pathways of coronavirus.

FIGS. 12A-12B show the concentration distributed in each organ (FIG. 12A) and the serum half-life (FIG. 12B) after injecting the antibody P2B-2FB or immunodisc in the form of scFv-Fc into mice to evaluate the pharmacodynamics of the nanodisc (immunodisc) of the present invention.

FIGS. 13A-13B show the concentration distributed in each organ (FIG. 13A) and the serum half-life (FIG. 13B) after injecting the sACE2-Fc or ACE2-immunodisc into mice to evaluate the pharmacodynamics of the nanodisc (ACE2-immunodisc) of the present invention containing angiotensin converting enzyme 2 (ACE2) as a virus receptor.

### BEST MODE

The present invention provides a nanodisc including a lipid bilayer being derived from phospholipid, having a flat disc-shaped bilayer structure, and including a hydrophilic group oriented outward and a hydrophobic group oriented inward, and a membrane scaffold protein (MSP) surrounding a side surface of the lipid bilayer where the hydrophobic group is exposed outside, wherein an Fc fragment is fused to the membrane scaffold protein (MSP) and protrudes outward from the side surface of the nanodisc.

A nanodisc has a structure in which the membrane scaffold protein (MSPs) surrounds the side surface of the lipid bilayer derived through from phospholipids a hydrophobic bond such that the hydrophilic groups of the phospholipids are oriented outward and the hydrophobic groups are oriented inward, and the lipid bilayer has a flat disc shape. Previous studies reported the wide use of nanodiscs as carriers for hydrophobic drugs and nanodiscs are also utilized in research on the structure and function of proteins.

The present inventors found in Korean Patent Nos. 10-2181991 and 10-2438720 that nanodiscs act as cell membrane mimics and puncture the outer envelopes of viruses, and found that nanodiscs are useful as antiviral agents for a wide range of viruses based on this mechanism.

During various studies to improve the antiviral efficacy of the nanodisc as described above, in the present invention, it was found that the nanodisc (immunodisc) of the present invention produced using a membrane scaffold protein (MSP) to which the Fc region of an antibody is fused increases the yield during the production process and exhibits excellent pharmacodynamics. Based on this finding, the present invention was completed.

In particular, it was found that the nanodisc of the present invention has a long half-life in the body. The following example shows that, when angiotensin converting enzyme (ACE2) is incorporated into the nanodisc, the half-life in the body can be increased by about 7 times. In other words, the nanodisc of the present invention is suitable for use as a carrier for hydrophobic drugs.

Meanwhile, in the present invention, for example, the phospholipid may include at least one selected from the group consisting of phosphatidylcholine, phosphatidylglycerol, phosphatidylethanolamine, phosphatidylserine, and phosphatidylinositol.

The phosphatidylcholine is, for example, DOPC (1,2-dioleoyl-sn-glycero-3-phosphocholine), DLPC (1,2-dilauroyl-sn-glycero-3-phosphocholine), DMPC (1,2-dimyristoyl-sn-glycero-3-phosphocholine), DPPC (1,2-dipalmitoyl-sn-glycero-3-phosphocholine), POPC (1-palmitoyl-2-oleoyl-sn-glycero-3-phosphocholine), C13PC, DDPC (1,2-didecanoyl-sn-glycero-3-phosphocholine), DSPC (1,2-distearoyl-sn-glycero-3-phosphocholine), DEPC (1,2-dierucoyl-sn-glycero-3-phosphocholine), DLOPC (1,2-dilinoleoyl-sn-glycero-3-phosphocholine), EPC (egg phosphatidylcholine), MSPC (1-myristoyl-2-stearoyl-sn-glycero-3-phosphocholine), PMPC (1-palmitoyl-2-myristoyl-sn-glycero-3-phosphocholine), PSPC (1-palmitoyl-2-stearoyl-sn-glycero-3-phosphocholine), SMPC (1-stearoyl-2-myristoyl-sn-glycero-3-phosphocholine) or SPPC (1-stearoyl-2-palmitoyl-sn-glycero-3-phosphocholine).

In addition, the phosphatidylglycerol is, for example, DMPG (1,2-dimyristoyl-sn-glycero-3 [phospho-rac-(1-glycerol)], DPPG (1,2-dipalmitoyl-sn-glycero-3 [phospho-rac-(1-glycerol)]), DSPG (1,2-distearoyl-sn-glycero-3 [phospho-rac-(1-glycerol)), POPG (1-palmitoyl-2-oleoyl-sn-glycero-3 [phospho-rac-(1-glycerol)]), DEPG (1,2-dierucoyl-sn-glycero-3 [phospho-rac-(1-glycerol)]), DLPG (1,2-dilauroyl-sn-glycero-3 [phospho-rac-(1-glycerol)]), DOPG (1,2-dioleoyl-sn-glycero-3 [phospho-rac-(1-glycerol)]) or DSPG (1,2-distearoyl-sn-glycero-3 [phospho-rac-(1-glycerol)]), the phosphatidylethanolamine is, for example, DMPE (1,2-dimyristoyl-sn-glycero-3-phosphoethanolamine), DPPE (1,2-dipalmitoyl-sn-glycero-3-phosphoethanolamine), DSPE (1,2-distearoyl-sn-glycero-3-phosphoethanolamine), DOPE (1,2-dioleoyl-sn-glycero-3-phosphoethanolamine), DEPE (1,2-dierucoyl-sn-glycero-3-phosphoethanolamine), DLPE (1,2-dilauroyl-sn-glycero-3-phosphoethanolamine) or POPE (1-palmitoyl-2-oleoyl-sn-glycero-3-phosphoethanolamine), the phosphatidylserine is, for example, DOPS (1,2-dioleoyl-sn-glycero-3-phosphoserine), DLPS (1,2-dilauroyl-sn-glycero-3-phosphoserine), DMPS (1,2-dimyristoyl-sn-glycero-3-phosphoserine), DPPS (1,2-dipalmitoyl-sn-glycero-3-phosphoserine), DSPS (1,2-distearoyl-sn-glycero-3-phosphoserine) or POPS, and the phosphatidylinositol is, for example, phosphatidylinositol-4-phosphate, phosphatidylinositol-4,5-bisphosphate, or phosphatidylinositol-3,4,5-tisphosphate

Meanwhile, in the present invention, the membrane scaffold protein (MSP) has a helix structure, is amphipathic and serves to surround the side surface of the lipid bilayer. An example of an amphipathic membrane scaffold protein is apolipoprotein. Apolipoprotein is a protein present specifically in plasma lipoproteins and is known to stabilize the structure of lipoproteins, activate enzymes involved in lipoprotein metabolism, and act as a ligand for lipoprotein receptors present on the cell surface. The apolipoprotein is, for example, apolipoprotein A1 (ApoA-I), apolipoprotein A2 (ApoA-2), apolipoprotein B (ApoB), apolipoprotein C (ApoC), apolipoprotein protein E (ApoE), MSP1 (membrane scaffold protein 1), MSP1D1, MSP1D2, MSP1E1, MSP1E2, MSP1E3, MSP1E3D1, MSP2, MSP2N1, MSP2N2, MSP2N3, or the like.

ApoA-I, provided as an example above, is known to be an ingredient of high-density lipoprotein (HDL), which plays a direct role in removing cholesterol from surrounding tissues and transporting the same to the liver or other lipoproteins. Apo-A1 is a 28 kDa single polypeptide consisting of 243 amino acids. Apo-A1 is a protein that has 8 repeating unit domains consisting of 11 amino acids or 22 amino acids, and 60 to 75% of alpha-helices in the secondary structure constituting HDL. In addition, like ApoA1, ApoE, is known to be involved in the transport of cholesterol and is a protein composed of a 33 kDa single polypeptide consisting of 299 amino acids.

In addition, in the present invention, a fraction of an apolipoprotein that maintains the helix structure and amphipathic characteristics of the apolipoprotein may be used. In other words, a part (fraction) of the apolipoprotein may be used instead of the entire apolipoprotein as long as the helix structure and amphipathic characteristics of the apolipoprotein are not lost.

Meanwhile, the nanodisc of the present invention may preferably further include a virus receptor. As used herein, the term “virus receptor” refers to a receptor that is capable of binding to a virus surface antigen and may be an antibody against a surface antigen, another cell membrane scaffold protein to which the surface antigen may bind, a compound to which the surface antigen may bind, or the like. In other words, since the nanodisc of the present invention further includes the virus receptor, it can exhibit an adhesion affinity to a virus. Meanwhile, the virus receptor may be incorporated into the nanodisc by forming a hydrophobic bond with the inside of the lipid bilayer of the nanodisc, or may be fused to the membrane scaffold protein.

In this case, the virus receptor is preferably angiotensin converting enzyme 2 or a compound having an end bound to sialic acid.

Several coronaviruses, including SARS-COV and SARS-CoV-2, are known to penetrate human cells using the angiotensin converting enzyme 2 (ACE2) as a receptor. In other words, by incorporating angiotensin converting enzyme 2, the ability to attach to coronaviruses, including SARS-CoV and SARS-COV-2, can be improved, or excellent antiviral efficacy against coronaviruses can be imparted.

The compound having an end bound to sialic acid refers to a compound or sialic acid complex that contains sialic acid at the end of the compound and thus has the ability to attach to a virus. Examples of such a compound include sialyllactose and ganglioside, and synthetic receptors thereof. Sialyllactose and ganglioside contain sialic acid at one end thereof and thus can enhance the ability to attach to influenza viruses or provide excellent antiviral efficacy against influenza viruses. Examples of the synthetic receptor include T3 (tetra-(2,3) sialyllactose-(ethylenglycol) 3-maleimido-(ethylenglycol) 3-stearyl, T6 (tetra-(2, 6) sialyllactose-(ethylenglycol) 3-maleimido-(ethylenglycol) 3-stearyl), 3slst (α(2, 3) sialyllactose-(ethylenglycol) 3-stearoyl), 6s1st (α(2, 6) sialyllactose-(ethylenglycol) 3-stearoyl), and the like.

The present invention provides a nanodisc including a lipid bilayer being derived from phospholipid, having a structure, and including a flat disc-shaped bilayer hydrophilic group oriented outward and a hydrophobic group oriented inward, a membrane scaffold protein (MSP) surrounding a side surface of the lipid bilayer where the hydrophobic group is exposed outside, and a virus receptor hydrophobically bonded to the lipid bilayer, wherein an Fc fragment is fused to the membrane scaffold protein (MSP) and protrudes outward from the side surface of the nanodisc, and a pharmaceutical composition for preventing or treating viral infection containing the same.

A nanodisc has a structure in which the membrane scaffold protein (MSPs) surrounds the side surface of the lipid bilayer derived from phospholipids with a hydrophobic bond, such that the hydrophilic groups of the phospholipids are oriented outward and the hydrophobic groups are oriented inward, and the lipid bilayer has a flat disc shape. Previous studies reported the wide use of nanodiscs as carriers for hydrophobic drugs and nanodiscs are also utilized in research on the structure and function of proteins.

The present inventors found in Korean Patent Nos. 10-2181991 and 10-2438720 that nanodiscs act as cell membrane mimics and puncture the outer envelopes of viruses, and found that nanodiscs are useful as antiviral agents for a wide range of viruses based on this mechanism.

Antibody treatments are mainly being developed for virus treatment. Nanodiscs have better antiviral efficacy, do not induce resistant viruses, and have high safety compared to virus treatment antibodies and similar technologies.

During various studies to improve the antiviral efficacy of the nanodisc as described above, in the present invention, it was found that the nanodisc (immunodisc) of the present invention produced using a membrane scaffold protein (MSP) to which the Fc region of an antibody is fused increases the yield during the production process and exhibits excellent antiviral activity and pharmacodynamics. Based on this finding, the present invention was completed.

In addition, the nanodisc of the present invention includes an Fc region of an antibody. Therefore, when a virus binds to the nanodisc of the present invention, macrophages can recognize the virus. As a result, the nanodisc can exhibit excellent antiviral efficacy.

Meanwhile, in the present invention, for example, the phospholipid may include at least one selected from the group consisting of phosphatidylcholine, phosphatidylglycerol, phosphatidylethanolamine, phosphatidylserine, and phosphatidylinositol.

The phosphatidylcholine is, for example, DOPC (1, 2-dioleoyl-sn-glycero-3-phosphocholine), DLPC (1, 2-dilauroyl-sn-glycero-3-phosphocholine), DMPC (1, 2-dimyristoyl-sn-glycero-3-phosphocholine), DPPC (1, 2-dipalmitoyl-sn-glycero-3-phosphocholine), POPC (1-palmitoyl-2-oleoyl-sn-glycero-3-phosphocholine), C13PC, DDPC (1, 2-didecanoyl-sn-glycero-3-phosphocholine), DSPC (1, 2-distearoyl-sn-glycero-3-phosphocholine), DEPC (1,2-dierucoyl-sn-glycero-3-phosphocholine), DLOPC (1, 2-dilinoleoyl-sn-glycero-3-phosphocholine), EPC (egg phosphatidylcholine), MSPC (1-myristoyl-2-stearoyl-sn-glycero-3-phosphocholine), PMPC (1-palmitoyl-2-myristoyl-sn-glycero-3-phosphocholine), PSPC (1-palmitoyl-2-stearoyl-sn-glycero-3-phosphocholine), SMPC (1-stearoyl-2-myristoyl-sn-glycero-3-phosphocholine) or SPPC (1-stearoyl-2-palmitoyl-sn-glycero-3-phosphocholine).

In addition, the phosphatidylglycerol is, for example, DMPG (1, 2-dimyristoyl-sn-glycero-3 [phospho-rac-(1-glycerol)], DPPG (1, 2-dipalmitoyl-sn-glycero-3 [phospho-rac-(1-glycerol)]), DSPG (1, 2-distearoyl-sn-glycero-3 [phospho-rac-(1-glycerol)), POPG (1-palmitoyl-2-oleoyl-sn-glycero-3 [phospho-rac-(1-glycerol)]), DEPG (1,2-dierucoyl-sn-glycero-3 [phospho-rac-(1-glycerol)]), DLPG (1, 2-dilauroyl-sn-glycero-3 [phospho-rac-(1-glycerol)]), DOPG (1,2-dioleoyl-sn-glycero-3 [phospho-rac-(1-glycerol)]) or DSPG (1, 2-distearoyl-sn-glycero-3 [phospho-rac-(1-glycerol)]), the phosphatidylethanolamine is, for example, DMPE (1,2-dimyristoyl-sn-glycero-3-phosphoethanolamine), DPPE (1,2-dipalmitoyl-sn-glycero-3-phosphoethanolamine), DSPE (1,2-distearoyl-sn-glycero-3-phosphoethanolamine), DOPE (1,2-dioleoyl-sn-glycero-3-phosphoethanolamine), DEPE (1,2-dierucoyl-sn-glycero-3-phosphoethanolamine), DLPE (1,2-dilauroyl-sn-glycero-3-phosphoethanolamine) or POPE (1-palmitoyl-2-oleoyl-sn-glycero-3-phosphoethanolamine), the phosphatidylserine is, for example, DOPS (1, 2-dioleoyl-sn-glycero-3-phosphoserine), DLPS (1, 2-dilauroyl-sn-glycero-3-phosphoserine), DMPS (1,2-dimyristoyl-sn-glycero-3-phosphoserine), DPPS (1, 2-dipalmitoyl-sn-glycero-3-phosphoserine), DSPS (1, 2-distearoyl-sn-glycero-3-phosphoserine) or POPS, and the phosphatidylinositol is, for example, phosphatidylinositol-4-phosphate, phosphatidylinositol-4, 5-bisphosphate, or phosphatidylinositol-4, 5-bisphosphate.

Meanwhile, in the present invention, the membrane scaffold protein (MSP) has a helix structure, is amphipathic and serves to surround the side surface of the lipid bilayer. An example of an amphipathic membrane scaffold protein is apolipoprotein. Apolipoprotein is a protein present specifically in plasma lipoproteins and is known to stabilize the structure of lipoproteins, activate enzymes involved in lipoprotein metabolism, and act as a ligand for lipoprotein receptors present on the cell surface. The apolipoprotein is, for example, apolipoprotein A1 (ApoA-I), apolipoprotein A2 (ApoA-2), apolipoprotein B (ApoB), apolipoprotein C (ApoC), apolipoprotein protein E (ApoE), MSP1 (membrane scaffold protein 1), MSP1D1, MSP1D2, MSP1E1, MSP1E2, MSP1E3, MSP1E3D1, MSP2, MSP2N1, MSP2N2, MSP2N3, or the like.

ApoA-I, provided as an example above, is known to be an ingredient of high-density lipoprotein (HDL), which plays a direct role in removing cholesterol from surrounding tissues and transporting the same to the liver or other lipoproteins. Apo-A1 is a 28 kDa single polypeptide consisting of 243 amino acids. Apo-A1 is a protein that has 8 repeating unit domains consisting of 11 amino acids or 22 amino acids, and 60 to 75% of alpha-helices in the secondary structure constituting HDL. In addition, like ApoA1, ApoE, is known to be involved in the transport of cholesterol and is a protein composed of a 33 kDa single polypeptide consisting of 299 amino acids.

In addition, in the present invention, a fraction of an apolipoprotein that maintains the helix structure and amphipathic characteristics of the apolipoprotein may be used. In other words, a part (fraction) of the apolipoprotein may be used instead of the entire apolipoprotein as long as the helix structure and amphipathic characteristics of the apolipoprotein are not lost.

Meanwhile, as used herein, the term “virus receptor” refers to a receptor that is capable of binding to a virus surface antigen and may be an antibody against a surface antigen, another cell membrane scaffold protein to which the surface antigen may bind, a compound to which the surface antigen may bind, or the like. In other words, the virus receptor further improves the ability of nanodisc to attach to viruses, thereby improving the antiviral efficacy.

In this case, the virus receptor is preferably angiotensin converting enzyme 2 or a compound having an end bound to sialic acid.

Several coronaviruses, including SARS-COV and SARS-CoV-2, are known to penetrate human cells using the angiotensin converting enzyme 2 (ACE2) as a receptor. In other words, by incorporating angiotensin converting enzyme 2, the rate at which coronaviruses including SARS-COV and SARS-COV-2, recognize the nanodisc of the present invention as cell membranes increases, thereby improving the antiviral efficacy against the coronaviruses.

It is known that the influenza virus uses sugar molecules containing sialic acid as receptors to penetrate human cells. When a compound having an end bound to sialic acid is contained, the antiviral efficacy against the influenza virus can be improved.

The compound having an end bound to sialic acid refers to a compound or sialic acid complex that contains sialic acid at the end of the compound and thus has the ability to attach to a virus. Examples of such compound include sialyllactose and ganglioside, and synthetic receptors thereof. Examples of the synthetic receptor include T3 (tetra-(2, 3) sialyllactose-(ethylenglycol) 3-maleimido-(ethylenglycol) 3-stearyl), T6 (tetra-(2, 6) sialyllactose-(ethylenglycol) 3-maleimido-(ethylenglycol) 3-stearyl), 3slst (α(2, 3) sialyllactose-(ethylenglycol) 3-stearoyl), 6slst (α(2, 6) sialyllactose-(ethylenglycol) 3-stearoyl), and the like.

Meanwhile, the pharmaceutical composition of the present invention may exhibit antiviral efficacy regardless of the type of virus. Therefore, in the present invention, the viral infection may be induced by at least one virus selected from Coronaviridae, Bunyaviridae, Filoviridae, Flaviviridae, Hepadnaviridae, Herpesviridae, Orthomyxoviridae, Poxviridae, Rhabdoviridae, Retroviridae and Togaviridae.

Meanwhile, the pharmaceutical composition of the present invention may further contain a pharmaceutically acceptable carrier, in addition to the active ingredient. The pharmaceutically acceptable carrier which may be contained in the present invention is commonly used in preparation and examples thereof include, but are not limited to, lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, and the like. In addition, the pharmaceutical composition of the present invention may further contain lubricants, humectants, sweeteners, flavoring agents, emulsifiers, suspending agents, preservatives and the like.

The pharmaceutical composition of the present invention may be administered orally or parenterally, for example, intrathecally, intravenously, subcutaneously, intradermally, intramuscularly, intraperitoneally, intrasternally, intratumorally, intranasally, intracerebrally, intracranially, intrapulmonarily, and intrarectally, but is not limited thereto.

That is, the pharmaceutically effective amount of the pharmaceutical composition of the present invention may vary depending on the formulation method, administration method, age, weight, gender, pathological condition, diet, administration time, administration route, excretion rate and reaction sensitivity of the patient. Ordinary skilled physicians can easily determine and prescribe an effective dosage (pharmaceutically effective amount) for desired treatment or prevention. According to a preferred embodiment of the present invention, the daily dose of the pharmaceutical composition of the present invention is about 0.0001 to about 100 mg/kg.

As used herein, the term “pharmaceutically effective amount” means an amount sufficient to prevent or treat the disease. As used herein, the term “prevention” refers to preventive or protective treatment of a disease or disease condition. As used herein, the term “treatment” refers to any action that reduces, inhibits, ameliorates or removes a disease or disease condition.

The pharmaceutical composition of the present invention is formulated into a unit dose form or packaged into a multiple dose container using a pharmaceutically acceptable carrier and/or excipient in accordance with a method that can be easily performed by those skilled in the art. The formulation may be prepared in a variety of forms, such as an oral drug or injection, may be in the form of a solution, suspension or emulsion in an oil or aqueous medium, or in the form of an extract, a powder, a suppository, a powder, a granule, a tablet or a capsule, and may further contain a dispersant or stabilizer.

Hereinafter, the present invention will be described in more detail with reference to the following examples, but the scope of the present invention is not limited to the examples and includes variations and technical concepts equivalent thereto.

### Example 1: Production of Nanodisc (Immunodisc) of Present Invention

In this example, a nanodisc (hereinafter referred to as “immunodisc”) of the present invention containing a membrane scaffold protein (MSP-Fc) fused with an Fc region of an antibody was produced.

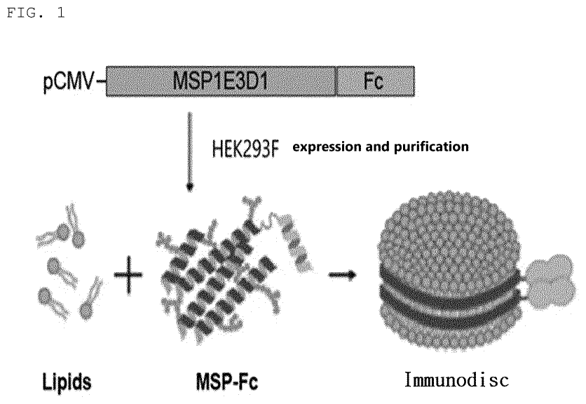

First, the MSP-Fc protein was expressed and purified in HEK293 cells, and then mixed with phospholipids to produce an immunodisc of the present invention (FIG. 1).

1-1. Production and Purification of Membrane Scaffold Protein (MSP-Fc) Fused with Fc Region of Antibody

To produce an MSP-Fc fusion protein, a plasmid including a sequence encoding MSP1E3D1 and a sequence encoding an Fc region of an antibody (SEQ ID NO: 2) was produced.

HEK293-soluble suspension cells were cultured under conditions of 37° C., 120 rpm, and 8% CO₂ to prepare 180 mL of a 1.1×10⁶ cells/mL culture medium. Then, 250 μg of the plasmid and 750 μg of PEI were was mixed with 20 mL of a culture medium and transfected into the prepared suspension cells. The cells were cultured for 96 hours in an incubator under conditions of 37° C., 120 rpm, and 8% CO₂, and centrifugation was performed at 8,000 g for 10 minutes, to remove the cells and only collect the supernatant. All the supernatant was allowed to flow into the protein G resin, and the protein was purified from the resin while pouring the elution buffer (0.1 M glycine, pH 2.8), and then the pH of the protein was adjusted to pH 7.4 by treatment with neutralization buffer (1 M Tris, pH 9.0) for stabilization.

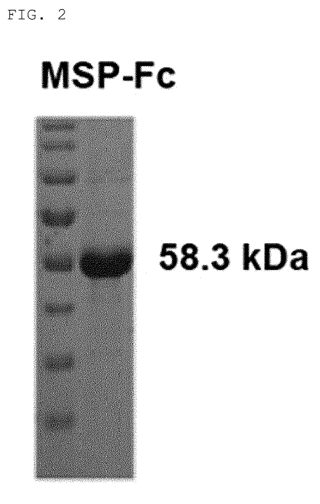

Then, the obtained protein was electrophoresed on an SDS-PAGE gel and the measured molecular weight was confirmed to be the same as the expected (58 kDa) (FIG. 2).

### 1-2. Production and Confirmation of Immunodisc

As a phospholipid, POPC (1-palmitoyl-2-oleoyl-sn-glycero-3-phosphocholine) was dissolved in chloroform to prepare 25 mg/mL of a POPC solution. 304 μL of the POPC solution in which the total lipid concentration was adjusted to 10 mM and the total volume was adjusted to 1 mL was transferred to a glass tube. Then, the solvent was removed using nitrogen gas and vacuum, and mixed with 1 mL of ND buffer (40 mM Tris-Cl, 300 mM NaCl, 0.5 mM EDTA, 50 mM NaC, pH 7.4) to induce hydration, and then sonication was performed at 55° C. for 30 minutes to obtain a POPC lipid film.

MSP-Fc and the PCPC lipid film were mixed such that the molar ratio of MSP-Fc to lipid was adjusted to 1:120. The resulting mixture was treated with the same amount of bio-beads at room temperature for 5 hours, the bio-beads were removed and the immunodisc was purified by size exclusion chromatography (SEC).

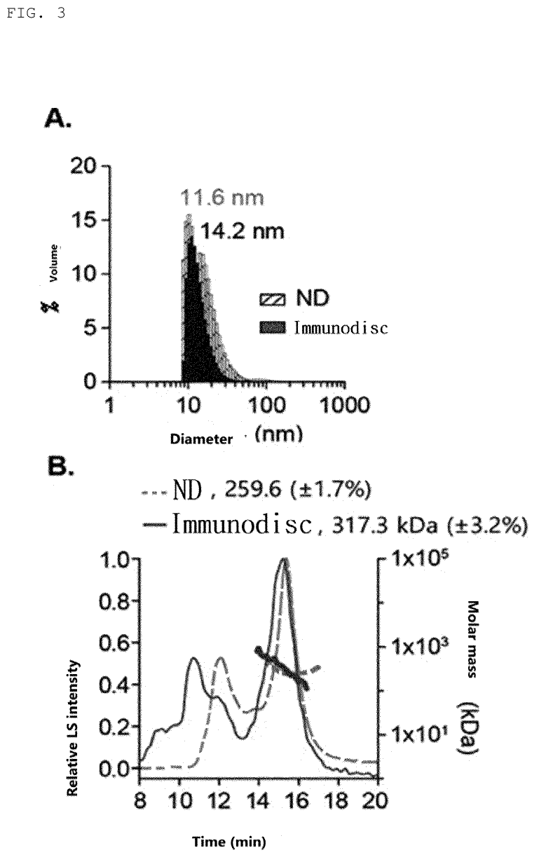

To determine whether or not the immunodisc was completely produced, the particle size was measured by dynamic light scattering (DLS). The result showed that the size of the immunodisc was 14.2 nm, which was approximately 2.5 nm larger than the conventional nanodisc (ND, 11.6 nm), which indicates that the immunodisc was completely produced (A of FIG. 3).

In addition, in order to distinguish the shape between liposomes and nanodisc having the equal size, the molecular weight of the conventional ND and the immunodisc was measured using a multi-angle light scattering (SEC-MALS) detector. The general ND had a molecular weight of 259.6 kDa with 1.7% error from the theoretical value of 255 kDa, whereas the immunodisc of the present invention had a molecular weight of 317.3 kDa with a 3.2% error from the theoretical value of 330 kDa, which indicates that the shape of nanodisc was well constructed (B of FIG. 3).

### 1-3. Production of Large Immunodisc

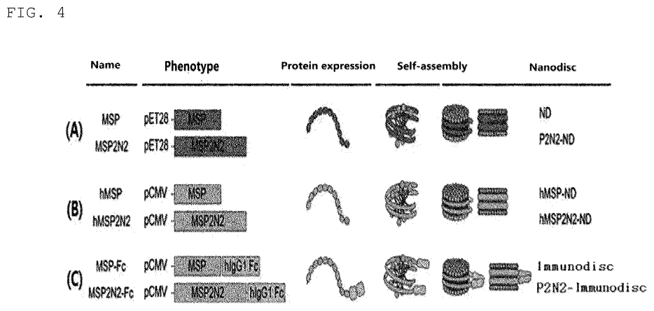

A nano disc with an increased size has advantages of being able to load more drugs therein or having stronger antiviral activity. In this example, a large membrane scaffold protein (MSP2N2-Fc) having an Fc region fused thereto was produced using a sequence encoding a membrane scaffold protein (MSP2N2) that may create a double disc size using two pieces of MSP and a sequence encoding an Fc region of an antibody (SEQ ID NO: 4) in accordance with the method of Example 1-1, and then a nanodisc was produced in accordance with the method of Example 1-2 and a large immunodisc (P2N2-immunodisc) was produced (FIG. 4).

### 1-4. Confirmation of Production Yield of General Immunodisc and Large Immunodisc (P2N2-Immunodisc)

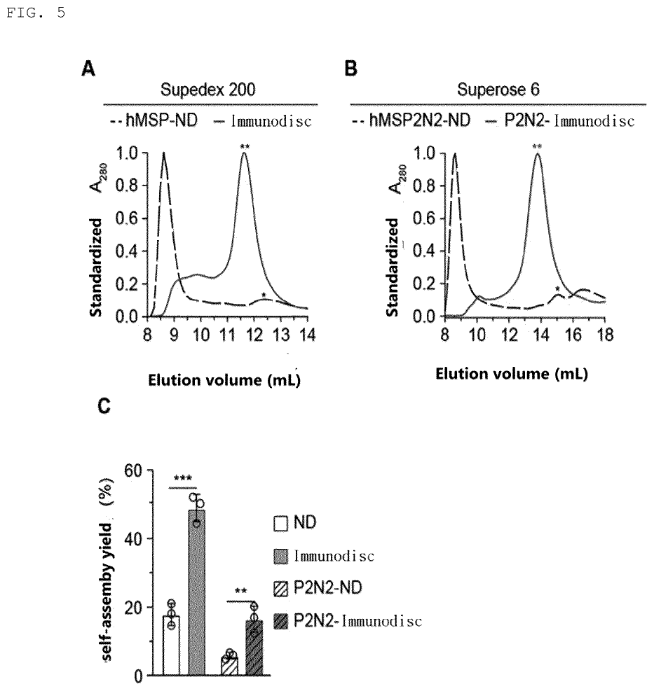

In this example, the production yield of the immunodiscs of the present invention was determined. To this end, the results of purification of the immunodiscs by size exclusion chromatography (SEC) in Examples 1-2 and 1-3 were compared and analyzed. Meanwhile, the columns for size exclusion chromatography (SEC) used herein were Superose 6 and Supedex 200, and the control group (hMSP, hMSP2N2) was used as a membrane scaffold protein without the Fc portion of an antibody produced from animal cells (HEK293-soluble suspension cells) for comparison (FIG. 5).

As can be seen from A and B of FIG. 5, when general MSP was used, most of it was eluted at about 9 mL of an elution volume and was produced in the form of an aggregate. On the other hand, most of the Fc-fused immunodisc was eluted at about 14 mL of an elution volume and was produced in the form of a monomer. The result shows that the Fc region of the membrane scaffold protein (MSP-Fc) fused with the Fc region of the antibody increased the production yield of the nanodisc.

C of FIG. 5 shows comparison in production yields calculated based on the results of A and B of FIG. 5, which indicates that the nanodisc produced using MSP-Fc exhibited an about 2.5 times increase in production yield compared to general MSP. In addition, the production yield of P2N2-ND was about 5%, whereas the production yield of P2N2-immunodisc was about 15%, which indicates that the P2N2-immunodisc exhibited an about 3 times increase in production yield.

1-5. Production of Immunodisc Including Synthetic Receptor Having End Fused with Sialic Acid as Virus Receptor

In this example, an immunodisc with improved antiviral efficacy was produced by incorporating a virus receptor into the immunodisc. To this end, an immunodisc was produced using ganglioside (GD1a), which is one of polysaccharides or glycolipids containing sialic acid used as an infection receptor of influenza virus, and a synthetic receptor based on sialyllactose (T3, tetra-(2, 3) sialyllactose-(ethylenglycol) 3-maleimido-(ethylenglycol) 3-stearyl; T6, tetra-(2, 6) sialyllactose-(ethylenglycol) 3-maleimido-(ethylenglycol) 3-stearyl; 3slst, a (2, 3) sialyllactose-(ethylenglycol) 3-stearoyl; 6slst, and a (2, 6) sialyllactose-(ethylenglycol) 3-stearoyl).

213 μL of 25 mg/mL POPC was transferred to a glass tube, 1,102 μL of 5 mg/mL GD1a, 333.3 μL of 10 mg/mL 3slst, 333.3 μL of 10 mg/ml 6slst, 1363.63 μL of 10 mg/ml T3 (tetra-2, 3-sialyllactose-stearic acid), and 1363.63 μL of 10 mg/mL of T6 (tetra-2, 6-sialyllactose-stearic acid) were added thereto and mixed to obtain a receptor-lipid mixture. The solvent was removed from the receptor-lipid mixture using nitrogen gas and vacuum to obtain a receptor-lipid film with a molar ratio of POPC: receptor of 7:3. The receptor-lipid film was hydrated with 1 mL of ND buffer (40 mM Tris-Cl, 300 mM NaCl, 0.5 mM EDTA, 50 mM NaC, pH 7.4) and sonicated at 55° C. for 30 minutes.

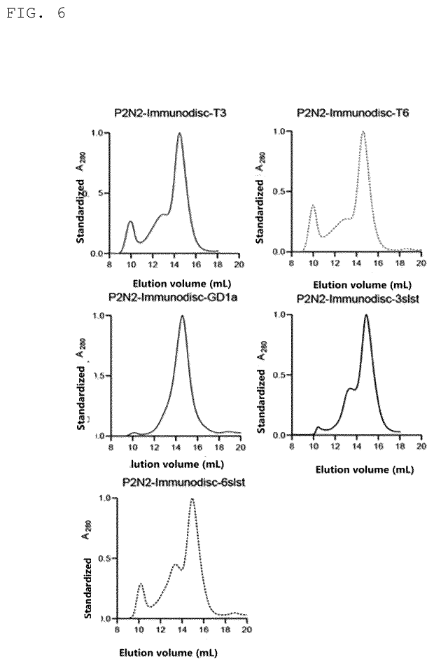

The membrane scaffold protein was mixed with the hydrated receptor-lipid film at a molar ratio of MSP2N2-Fc to lipid of 1:300, and the resulting mixture was treated with the same amount of bio-beads as the entire mixture at room temperature for 5 hours, and then the bio-beads were removed and purified through size exclusion chromatography (SEC) to obtain the virus receptor-Immunodisc. The result showed that large immunodiscs (P2N2-Immunodisc-T3, P2N2-Immunodisc-T6, P2N2-Immunodisc-GD1a, P2N2-Immunodisc-6slst, P2N2-Immunodisc-3slst) including the viral receptor in the form of a monomer were produced at approximately 14 to 15 mL of an elution volume (FIG. 6).

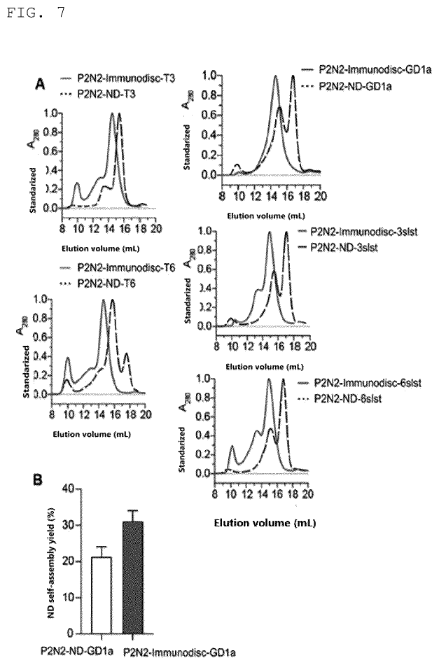

Meanwhile, the result of size exclusion chromatography (SEC) shows that the immunodisc including a synthetic viral receptor having an end bound to sialic acid bound had a high production yield of 1.5 times compared to the nanodisc produced using a general membrane scaffold protein (MSP) (FIG. 7).

### 1-6. Production of Immunodisc Containing Angiotensin Converting Enzyme 2 (ACE) as Virus Receptor

In this example, ACE2 (angiotensin converting enzyme 2, SEQ ID NO: 5), which is used as an infection receptor of coronavirus, was incorporated into an immunodisc, to produce an immunodisc (ACE2-immunodisc) with improved antiviral efficacy.

As lipids, 1-palmitoyl-2-oleoyl-sn-glycero-3-phosphocholine (POPC) and 1, 2-dioleoyl-sn-glycero-3-phospho-L-serine (DOPS) were dissolved in chloroform to prepare lipid solutions at concentrations of 25 mg/ml and 10 mg/ml, respectively. Then, 243 μL of a POPC solution and 65 μL of a DOPS solution dissolved in nanodisc (ND) buffer (40 mM Tris-Cl, 300 mM NaCl, 0.5 mM EDTA, 50 mM NaC, pH 7.4) such that the total lipid concentration was 10 mM, the total lipid volume was 1 mL, and at the same time, and the molar ratio of POPC: DOPS was 8:2, were transferred to a glass tube. Then, nitrogen gas was added thereto and the result was allowed to stand in a vacuum for at least 4 hours to remove the solvent and thereby to obtain a lipid film. 1 mL of the ND buffer was mixed with the obtained lipid film to hydrate the lipid film and ultrasonication was performed at 55° C. for 30 minutes to obtain a lipid suspension in which the lipids were homogeneously dispersed.

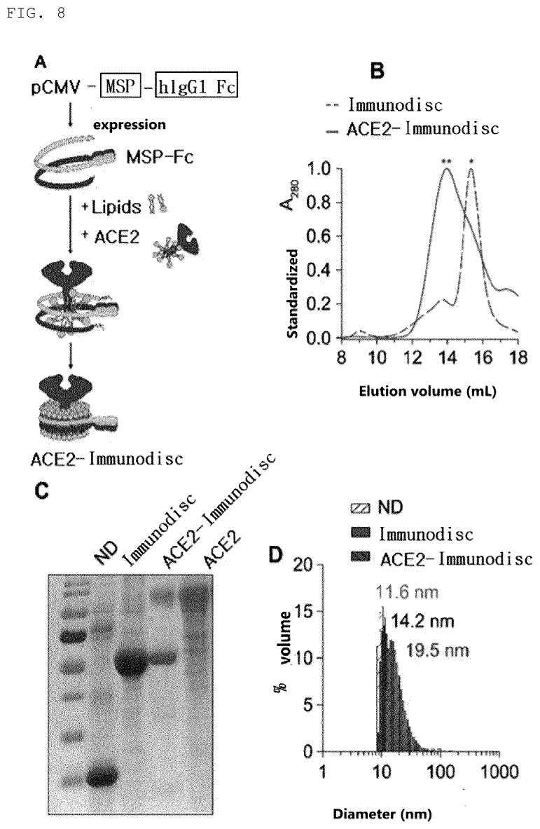

ACE2 (molecular weight 94.2 kDa): MSP-Fc (molecular weight 57 kDa): lipid were mixed at a molar ratio of 0.5:1:120. Then, the resulting mixture was treated with the same amount of bio-beads as the total mixture twice, more specifically, once at room temperature for 5 hours and once at 4° C. for 16 hours, to produce an ACE2-containing immunodisc (ACE2-immunodisc) through a self-assembly process (A of FIG. 8).

The produced ACE2-containing immunodisc was analyzed by size exclusion chromatography (SEC). The result shows that the ACE2-containing immunodisc was observed in an elution volume of 13 to 14 mL (B of FIG. 8). As a result of SDS-PAGE analysis of fractions with 13 to 14 mL of an elution volume, bands of ACE2 and the immunodisc were observed (C of FIG. 8). This indicates that that 13 to 14 mL of the monomer corresponds to the immunodisc containing ACE2. In addition, the result of DLS analysis shows that the diameter of the immunodisc containing ACE2 (19.5 nm) was 5.3 nm larger than the diameter of the immunodisc not containing ACE2 (14.2 nm), which indicates that the ACE2-immunodisc was successfully formed (D of FIG. 8).

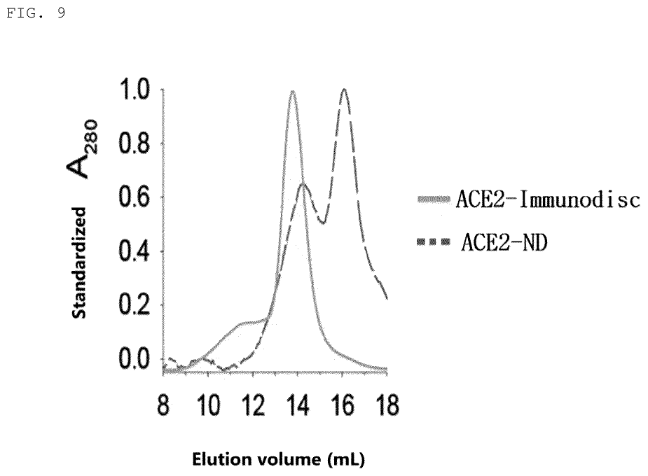

Meanwhile, the production yield of the nanodisc according to the present invention was compared with that of the nanodisc produced using a general membrane scaffold protein (MSP) using size exclusion chromatography (SEC). The result shows that the immunodisc was formed as a monomer (elution volume 13-14 mL) even if they contained ACE2, whereas the ACE2-nanodisc (elution volume 14 mL) and the nanodisc not containing ACE2 (elution volume 16 mL) were produced from the general nanodisc. The results show that conventional nanodisc often did not contain ACE2, where the immunodisc was uniformly formed in the form of a disc containing ACE2. This shows that, when a membrane scaffold protein (Fc-MSP) containing an Fc portion was used, the ACE2 loading ability was improved, and nanodiscs could be produced with a higher yield (FIG. 9).

### Example 2: Evaluation of Antiviral Efficacy of Immunodisc

In this example, whether or not the immunodisc produced in Example 1 exhibits excellent antiviral efficacy was determined.

2-1. Confirmation of Antiviral Efficacy of Immunodisc Including Virus Receptor Having End Bound with Sialic Acid

The antiviral efficacy of the immunodisc including synthetic virus receptor having end bound with sialic acid produced in Example 1-5 was compared with the nanodisc by performing experiment of neutralization efficacy against influenza virus.

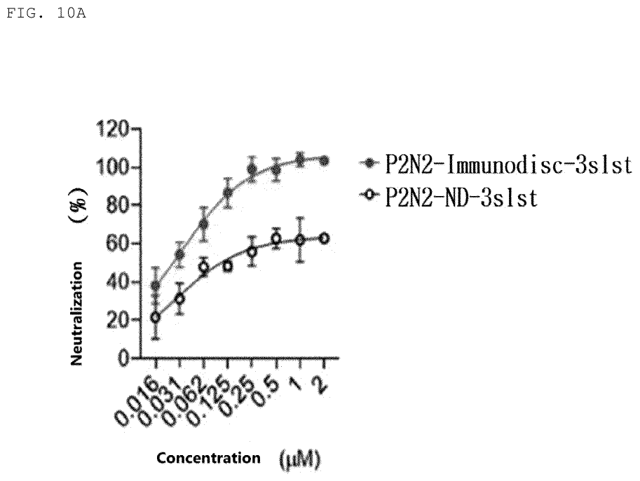

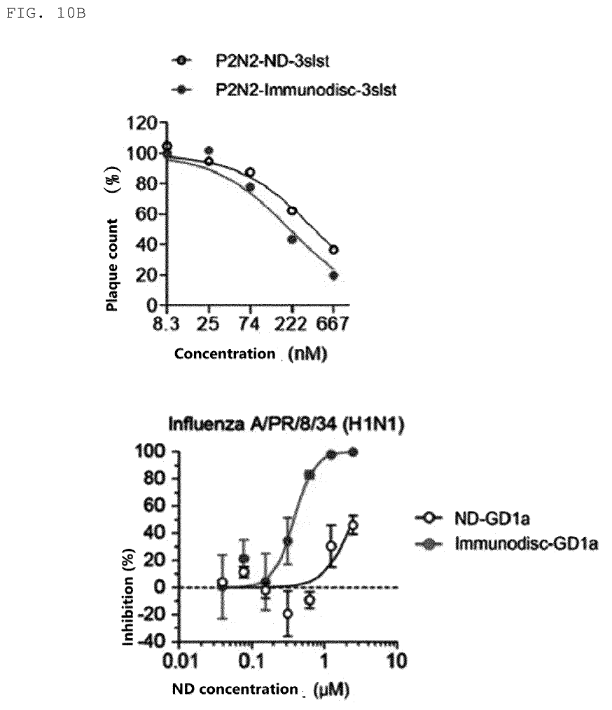

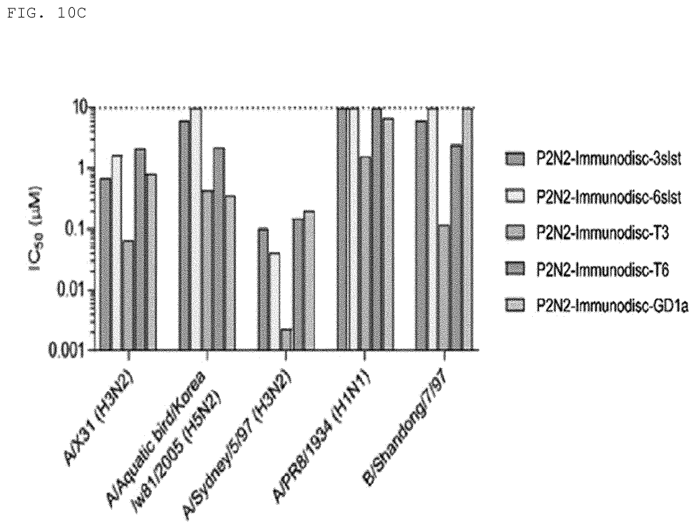

Specifically, 1×104 MDCK cells were seeded into each well of a 96-well black plate and cultured for 24 hours. The final concentration of A/Sydney/5/97 H3N2 influenza virus was adjusted to 0.01 MOI and the final concentration of the immunodisc was adjusted to 2 μM, and the MDCK cells washed twice with PBS were treated therewith. After 24 hours, the cells were treated with MUNANA at a final concentration of 100 μM and incubated at 37° C. for 1 hour. Then, the fluorescence at an excitation wavelength of 355 nm and an emission wavelength of 460 nm was measured using a spectrophotometer and the neutralization efficacy was compared (FIG. 10A).

As can be seen from FIG. 10A, the immunodisc exhibited 100% neutralization efficacy starting from 250 nM and 58% neutralization efficacy at 30 nM. On the other hand, it can be seen that the general nanodisc had 50% neutralization efficacy at 250 nM and 30% neutralization efficacy at 30 nM. The results show that the immunodisc has improved antiviral efficacy than the general nanodisc.

In addition, the influenza virus was subjected to plaque reduction analysis and the antiviral efficacy of the immunodisc was compared with that of the nanodisc including a synthetic virus receptor having an end bound to sialic acid.

Specifically, 1×10⁶ cells of MDCK cells were seeded into each well of a 6-well plate and cultured for 24 hours. The MDCK cells were washed twice with PBS and then treated with 100 PFU/mL of A/Sydney/7/97 H3N2 influenza virus. The culture medium was removed from each well and the cells were treated with 1.5 ml of agarose solution (HEPES 25 mM, sodium bicarbonate 22 mM, DMEM, 1% agarose, pH 7.4) including the nanodisc at concentrations of 8.3, 25, 74, 222, or 667 nM, and then were allowed to solidify at room temperature. Then, the cells were cultured for 3 days in a 5% CO₂ incubator at 37° C., and the number of formed plaques s was measured. Meanwhile, an experimental group using an agarose solution containing phosphate buffered saline (PBS) was used as a control group (FIG. 10B).

As can be seen from B in FIG. 10B, when comparing the nanodisc containing 3slst with the immunodisc, the immunodisc had 60% infection inhibition at 222 nM. On the other hand, it can be seen that the general nanodisc had 40% infection inhibition at 222 nM. In addition, it can be seen that, when comparing the immunodisc with the nanodisc having the Ganglioside GD1a receptor, the immunodisc had antiviral activity that was about 10 times stronger. The results indicate that the immunodisc has improved antiviral efficacy than the general nanodisc.

In addition, neutralization efficacy experiments were conducted on various influenza viruses to determine whether or not the immunodisc had broad-spectrum antiviral efficacy.

Specifically, 1×10⁴ MDCK cells were seeded onto each well of a 96-well black plate and cultured for 24 hours. The A/X31 H3N2, A/Aquatic bird/Korea/w81/2005 H5N2, A/Sydney/5/97 H3N2, A/Puerto Rico/1934 H1N1, and B/Shandong/7/97 influenza viruses were mixed such that the final concentration was adjusted to a MOI of 0.005-0.01 and the final concentration of the immunodisc was adjusted to 1 μM, and then the cells washed twice with PBS were treated with. After 24 hours, the cells were treated with MUNANA to a final concentration of 100 μM and incubated at 37° C. for 1 hour. The fluorescence at an excitation wavelength of 355 nm and an emission wavelength of 460 nm was measured using a spectrophotometer and the neutralization efficacy was compared (FIG. 10C).

As can be seen from FIG. 10C, the immunodisc had antiviral efficacy against all influenza viruses with different types of envelopes. Meanwhile, the immunodisc containing T3 as a virus receptor had the lowest IC50 concentration and showed the best antiviral efficacy.

### 2-2. Confirmation of Antiviral Efficacy of Immunodiscs Including ACE2 (Angiotensin Converting Enzyme 2)

A CPE inhibition assay using authentic SARS-COV-2 virus was performed to compare the antiviral efficacy of the nanodisc with that of the immunodisc including ACE2 produced in Example 1-6.

Specifically, Calu-3 and Vero E6 cells were seeded into each well of a 96-well cell culture plate at a concentration of 2×10⁵ cells/mL (100 μL each) and cultured in a 5% CO₂ incubator at 37° C. for 24 hours. The medium was removed from the cells and the residue was mixed with 50 μL of 100 TCID50 (tissue cell infectious dose) of SARS-COV-2 virus and 50 μL of antiviral agent diluted at various concentrations. Each well was treated with 100 μL of the mixture to induce infection at 37° C. for 1 hour. Then, the supernatant was removed and 100 μL of the medium containing the antiviral agent at each concentration was seeded to each well, followed by culturing in a 5% CO₂ incubator at 37° C. for 48 to 72 hours.

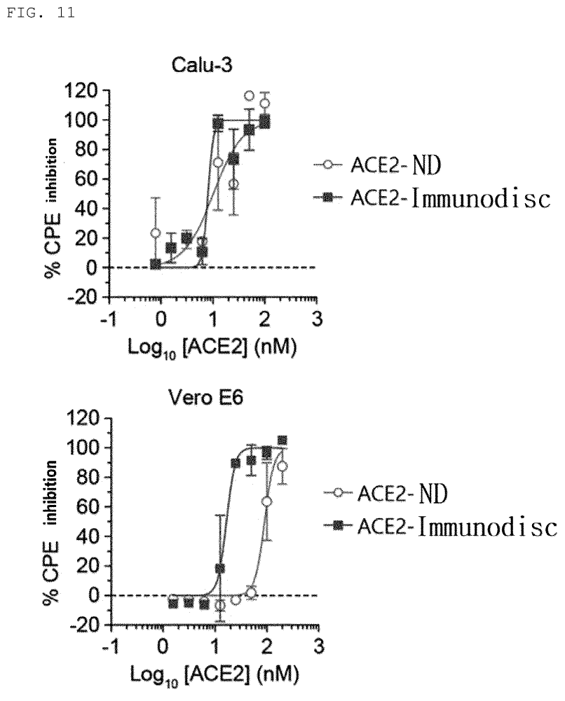

The cell supernatant was removed, 100 μL of 4% formaldehyde was seeded to each well, reaction was performed at 25° C. for 1 hour to induce immobilization, and then 100 μL of a 0.5% crystal violet solution was seeded to each well and the cells were stained therewith at 25° C. for 1 hour. The crystal violet solution was removed, each well was treated with 100 μL of methanol to dissolve the dried crystal violet, the absorbance at a wavelength of 570 nm was measured using a spectrophotometer and the cytopathic effect was determined (FIG. 11).

As can be seen from FIG. 11, the antiviral efficacy of the ACE2-containing immunodisc is superior to that of the ACE2-containing nanodisc. Meanwhile, SARS-COV-2 infects cells through two major routes. The first route is an endocytic pathway infection, which is an infection route in cases where only ACE2 is expressed on the surface, such as in Vero E6 cells. The second route is direct fusion infection, which is an infection route in cases where ACE2 and TMPRSS2 (SEQ ID NO.: 6) are simultaneously expressed, such as in Calu-3 cells.

### Example 3: Evaluation of Pharmacodynamics of Immunodisc

In this example, the pharmacodynamics of the immunodisc produced in Example 1 were determined. To this end, the immunodisc was injected into mice and the distribution in the mice was measured at each time.

### 3-1. Evaluation of Pharmacodynamics of Immunodisc

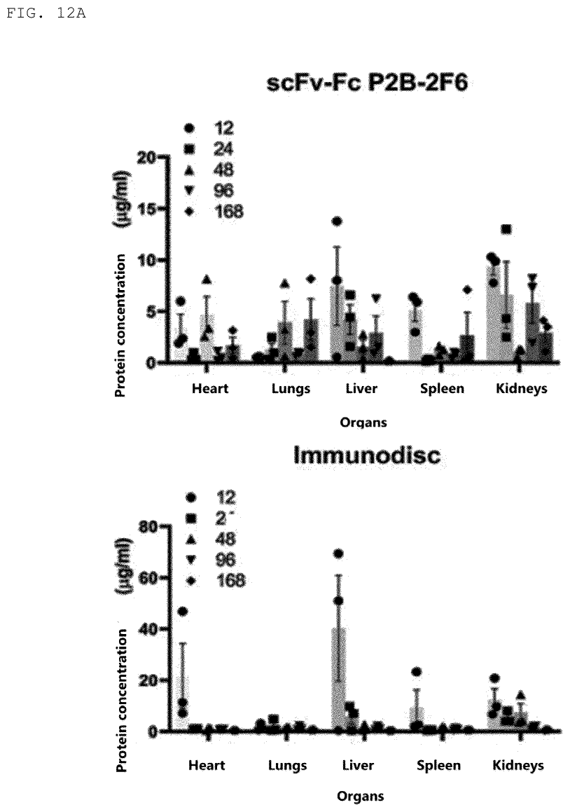

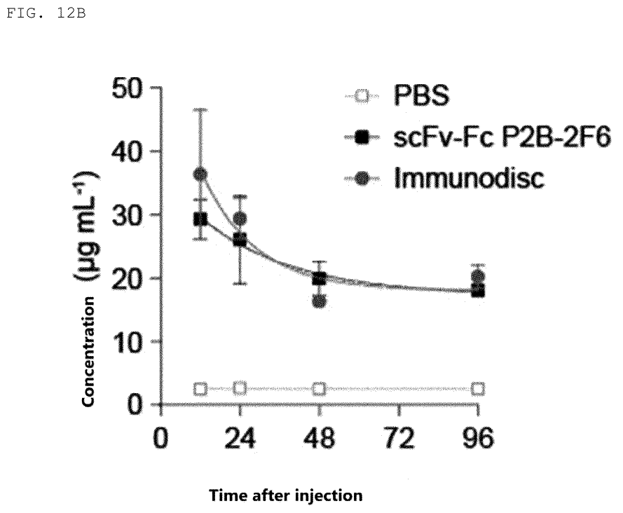

ScFv-Fc antibody P2B-2FB (SEQ ID NO: 7) or an immunodisc was injected into the tail vein of a mouse, blood and each organ (heart, lungs, liver, spleen, kidney) were collected from each experimental group mouse at 12, 24, 48, 96, and 168 hours, and then ground, and the concentration of the antibody and immunodisc injected into the mice was determined by ELISA (FIGS. 12A-12B).

FIG. 12A shows the concentration of the antibody or immunodisc distributed to each organ. The antibody was found to be widely distributed to various organs. On the other hand, the immunodisc of the present invention was concentrated in the liver. The result is considered to be because the protein-lipid structure of the immunodisc is similar to the structure of the HDL recognized by hepatocytes.

FIG. 12B shows the concentration of antibody or immunodisc in mouse serum, and indicates that both the antibody and the immunodisc of the present invention have similar half-lives. The results indicate that the immunodisc has excellent pharmacodynamics similar to the antibody.

### 3-2. Evaluation of Pharmacodynamics of Immunodiscs Containing ACE2

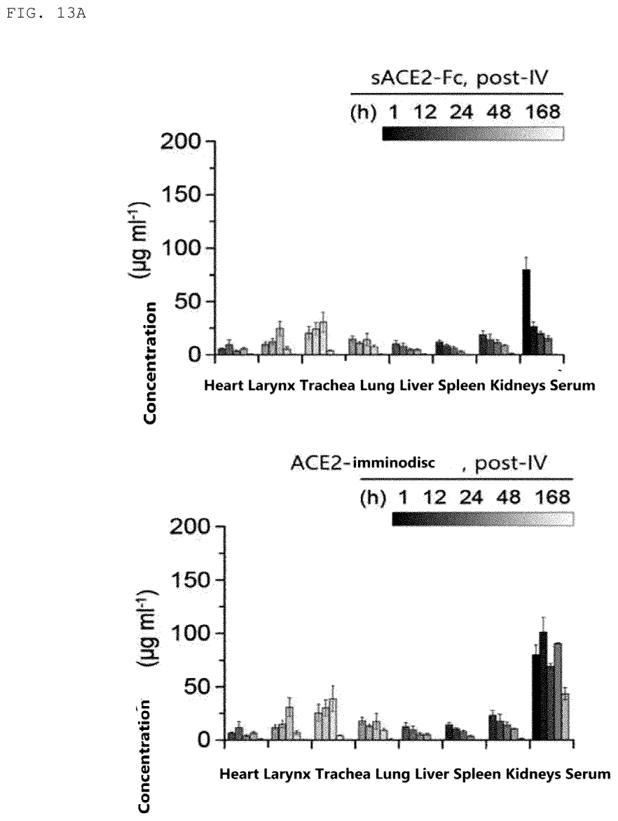

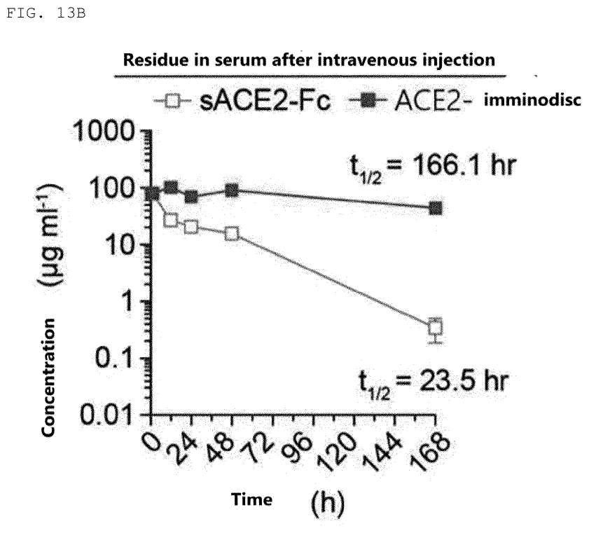

4.5 mg/kg of the immunodisc containing sACE2-Fc (SEQ ID NO: 8) or ACE2 was injected into the tail vein of mice, blood and each organ (heart, larynx, trachea, lungs, liver, spleen, kidney) were collected from each experimental group mouse after 12, 24, 48, 96, and 168 hours, and then ground, and the concentrations of the antibodies and immunodiscs injected into the mice were determined through ELISA (FIGS. 13A-13B).

FIG. 13A shows the concentration of immunodiscs containing sACE2-Fc or ACE2 distributed in each organ. It was found that most of the immunodisc containing sACE2-Fc and ACE2 was present in the serum and moved predominantly to the upper respiratory tract including the larynx and trachea.

FIG. 13B shows the concentration of immunodiscs containing sACE2-Fc or ACE2 in mouse serum. It was found that the half-life of the immunodisc containing ACE2 was 166.1 hours, which was lower than the half-life of sACE2-Fc (23.5 hr).

In particular, the half-life of conventional protein-based nanodiscs reported in the literature ranges from 0.5 to 2 hours to up to 60 hours (Denisov, Ilia G., and Stephen G. Sligar. “Nanodiscs in membrane biochemistry and biophysics.” Chemical reviews 117.6 (2017): 4669-4713.; Simon-Gracia, Lorena, et al. “Paclitaxel-Loaded Cationic Fluid Lipid Nanodiscs and Liposomes with Brush-Conformation PEG Chains Penetrate Breast Tumors and Trigger Caspase-3 Activation.” ACS Applied Materials & Interfaces (2022); Park, Hyun-Ji, et al. “High-density lipoprotein-mimicking nanodiscs carrying peptide for enhanced therapeutic angiogenesis in diabetic hindlimb ischemia.” Biomaterials 161 (2018): 69-80.) and almost no nanodiscs were observed in the blood after 96 hours (Hyun-Ji Park, 2018). It was found that the ACE2-containing immunodisc of the present invention maintained almost the same concentration without a significant difference from the initial immunodisc concentration even after 48 hours, and it was found that the immunodisc in the serum maintained a high concentration of about 40% of the initial concentration even after 168 hours.

1. A nanodisc comprising:
a lipid bilayer being derived from phospholipid, having a flat disc-shaped bilayer structure, and including a hydrophilic group oriented outward and a hydrophobic group oriented inward; and
a membrane scaffold protein (MSP) surrounding a side surface of the lipid bilayer where the hydrophobic group is exposed outside,
wherein an Fc fragment is fused to the membrane scaffold protein (MSP) and protrudes outward from a side surface of the nanodisc.

2. The nanodisc according to claim 1, wherein the phospholipid comprises at least one selected from the group consisting of phosphatidylcholine, phosphatidylserine, phosphatidylethanolamine, phosphatidylglycerol and phosphatidylinositol.

3. The nanodisc according to claim 1, wherein the phospholipid comprises at least one selected from POPC (1-palmitoyl-2-oleoyl-sn-glycero-3-phosphocholine), DOPS (1, 2-dioleoyl-sn-glycero-3-phospho-L-serine), and POPE (1-palmitoyl-2-oleoyl-sn-glycero-3-phosphoethanolamine).

4. The nanodisc according to claim 1, wherein the membrane scaffold protein (MSP) is an amphipathic protein having a helix structure.

5. The nanodisc according to claim 4, wherein the membrane scaffold protein is apolipoprotein or a fraction of apolipoprotein that maintains the helix structure and amphipathic characteristics of the apolipoprotein.

6. A nanodisc comprising:
a lipid bilayer being derived from phospholipid, having a flat disc-shaped bilayer structure, and including a hydrophilic group oriented outward and a hydrophobic group oriented inward;
a membrane scaffold protein (MSP) surrounding a side surface of the lipid bilayer where the hydrophobic group is exposed outside; and
a virus receptor hydrophobically bonded to the lipid bilayer,
wherein an Fc fragment is fused to the membrane scaffold protein (MSP) and protrudes outward from the side surface of the nanodisc.

7. The nanodisc according to claim 6, wherein the phospholipid comprises at least one selected from the group consisting of phosphatidylcholine, phosphatidylserine, phosphatidylethanolamine, phosphatidylglycerol and phosphatidylinositol.

8. The nanodisc according to claim 6, wherein the phospholipid comprises at least one selected from POPC (1-palmitoyl-2-oleoyl-sn-glycero-3-phosphocholine), DOPS (1, 2-dioleoyl-sn-glycero-3-phospho-L-serine), and POPE (1-palmitoyl-2-oleoyl-sn-glycero-3-phosphoethanolamine).

9. The nanodisc according to claim 6, wherein the membrane scaffold protein (MSP) is an amphipathic protein having a helix structure.

10. The nanodisc according to claim 9, wherein the membrane scaffold protein is apolipoprotein or a fraction of apolipoprotein that maintains the helix structure and amphipathic characteristics of the apolipoprotein.

11. The nanodisc according to claim 6, wherein the virus receptor is angiotensin converting enzyme 2 or a compound having an end bound to sialic acid.

12. A pharmaceutical composition for preventing or treating viral infection comprising:
a nanodisc comprising:
a lipid bilayer being derived from phospholipid, having a flat disc-shaped bilayer structure, and including a hydrophilic group oriented outward and a hydrophobic group oriented inward;
a membrane scaffold protein (MSP) surrounding a side surface of the lipid bilayer where the hydrophobic group is exposed outside; and
a virus receptor hydrophobically bonded to the lipid bilayer,
wherein an Fc fragment is fused to the membrane scaffold protein (MSP) and protrudes outward from the side surface of the nanodisc.

13. The pharmaceutical composition according to claim 12, wherein the phospholipid comprises at least one selected from the group consisting of phosphatidylcholine, phosphatidylserine, phosphatidylethanolamine,
phosphatidylglycerol and phosphatidylinositol.

14. The pharmaceutical composition according to claim 12, wherein the phospholipid comprises at least one selected from POPC (1-palmitoyl-2-oleoyl-sn-glycero-3-phosphocholine), DOPS (1, 2-dioleoyl-sn-glycero-3-phospho-L-serine), and POPE (1-palmitoyl-2-oleoyl-sn-glycero-3-phosphoethanolamine).

15. The pharmaceutical composition according to claim 12, wherein the membrane scaffold protein (MSP) is an amphipathic protein having a helix structure.

16. The pharmaceutical composition according to claim 15, wherein the membrane scaffold protein is apolipoprotein or a fraction of apolipoprotein that maintains the helix structure and amphipathic characteristics of the apolipoprotein.

17. The pharmaceutical composition according to claim 12, wherein the virus receptor is angiotensin converting enzyme 2 or a compound having an end bound to sialic acid.

18. The pharmaceutical composition according to claim 12, wherein the virus comprises at least one virus selected from Coronaviridae, Bunyaviridae, Filoviridae, Flaviviridae, Hepadnaviridae, Herpesviridae, Orthomyxoviridae, Poxviridae, Rhabdoviridae, Retroviridae and Togaviridae.


SEQUENCE LISTING (8 sequences)

SEQ ID NO: 1 - DNA, 834 bp, synthetic construct
  source 1..834: mol_type = other DNA
ggtcatcatcatcatcatcatcacgattatgatattcctactactgagaatttgtattttcagggttctaccttcagtaaacttcgcgaacaactgggccccgtgacgcaggaattctgggacaacctggaaaaagaaaccgagggactgcgtcaggaaatgtccaaagatttagaagaggtgaaggccaaggttcagccatatctcgatgactttcagaaaaaatggcaggaagagatggaattatatcgtcaaaaggtggaaccgctgcgtgcggaactgcaagagggggcacgccaaaaactccatgagctccaagagaagctcagcccattaggcgaagaaatgcgcgatcgcgcccgtgcacatgttgatgcactccggactcatttggcgccatatctcgatgactttcagaaaaaatggcaggaagagatggaattatatcgtcaaaaggtggaaccgctgcgtgcggaactgcaagagggggcacgccaaaaactccatgagctccaagagaagctcagcccattaggcgaagaaatgcgcgatcgcgcccgtgcacatgttgatgcactccggactcatttggcgccgtattcggatgaacttcgccagcgtttggccgcacgtctcgaggcgctgaaagaaaacgggggtgcccgcttggctgagtaccacgcgaaagcgacagaacacctgagcaccttgagcgaaaaagcgaaaccggcgctggaagatctacgccagggcttattgcctgttcttgagagctttaaagtcagttttctgtcagctctggaagaatatactaaaaagctgaatacccagtaa

SEQ ID NO: 2 - DNA, 1527 bp, synthetic construct
  source 1..1527: mol_type = other DNA
atggagacagacacaatcctgctatgggtgttgttattactcgcggcccagccggccatgaccttcagtaaacttcgcgaacaactgggccccgtgacgcaggaattctgggacaacctggaaaaagaaaccgagggactgcgtcaggaaatgtccaaagatttagaagaggtgaaggccaaggttcagccatatctcgatgactttcagaaaaaatggcaggaagagatggaattatatcgtcaaaaggtggaaccgctgcgtgcggaactgcaagagggggcacgccaaaaactccatgagctccaagagaagctcagcccattaggcgaagaaatgcgcgatcgcgcccgtgcacatgttgatgcactccggactcatttggcgccatatctcgatgactttcagaaaaaatggcaggaagagatggaattatatcgtcaaaaggtggaaccgctgcgtgcggaactgcaagagggggcacgccaaaaactccatgagctccaagagaagctcagcccattaggcgaagaaatgcgcgatcgcgcccgtgcacatgttgatgcactccggactcatttggcgccgtattcggatgaacttcgccagcgtttggccgcacgtctcgaggcgctgaaagaaaacgggggtgcccgcttggctgagtaccacgcgaaagcgacagaacacctgagcaccttgagcgaaaaagcgaaaccggcgctggaagatctacgccagggcttattgcctgttcttgagagctttaaagtcagttttctgtcagctctggaagaatatactaaaaagctgaatacccagggcccgggaggccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 3 - DNA, 1176 bp, synthetic construct
  source 1..1176: mol_type = other DNA
atgggtcatcatcatcatcatcatcacgattatgatattcctactactgagaatttgtattttcagggttctaccttcagtaaacttcgcgaacaactgggccccgtgacgcaggaattctgggacaacctggaaaaagaaaccgagggactgcgtcaggaaatgtccaaagatttagaagaggtgaaggccaaggttcagccatatctcgatgactttcagaaaaaatggcaggaagagatggaattatatcgtcaaaaggtggaaccgctgcgtgcggaactgcaagagggggcacgccaaaaactccatgagctccaagagaagctcagcccattaggcgaagaaatgcgcgatcgcgcccgtgcacatgttgatgcactccggactcatttggcgccgtattcggatgaacttcgccagcgtttggccgcacgtctcgaggcgctgaaagaaaacgggggtgcccgcttggctgagtaccacgcgaaagcgacagaacacctgagcaccttgagcgaaaaagcgaaaccggcgctggaagatctacgccagggcttattgcctgttcttgagagctttaaagtcagttttctgtcagctctggaagaatatactaaaaagctgaatacccagggtacccccgtgacgcaggaattctgggacaacctggaaaaagaaaccgagggactgcgtcaggaaatgtccaaagatttagaagaggtgaaggccaaggttcagccatatctcgatgactttcagaaaaaatggcaggaagagatggaattatatcgtcaaaaggtggaaccgctgcgtgcggaactgcaagagggggcacgccaaaaactccatgagctccaagagaagctcagcccattaggcgaagaaatgcgcgatcgcgcccgtgcacatgttgatgcactccggactcatttggcgccgtattcggatgaacttcgccagcgtttggccgcacgtctcgaggcgctgaaagaaaacgggggtgcccgcttggctgagtaccacgcgaaagcgacagaacacctgagcaccttgagcgaaaaagcgaaaccggcgctggaagatctacgccagggcttattgcctgttcttgagagctttaaagtcagttttctgtcagctctggaagaatatactaaaaagctgaatacccag

SEQ ID NO: 4 - DNA, 1869 bp, synthetic construct
  source 1..1869: mol_type = other DNA
atggagacagacacaatcctgctatgggtgttgttattactcgcggcccagccggccatgaccttcagtaaacttcgcgaacaactgggccccgtgacgcaggaattctgggacaacctggaaaaagaaaccgagggactgcgtcaggaaatgtccaaagatttagaagaggtgaaggccaaggttcagccatatctcgatgactttcagaaaaaatggcaggaagagatggaattatatcgtcaaaaggtggaaccgctgcgtgcggaactgcaagagggggcacgccaaaaactccatgagctccaagagaagctcagcccattaggcgaagaaatgcgcgatcgcgcccgtgcacatgttgatgcactccggactcatttggcgccgtattcggatgaacttcgccagcgtttggccgcacgtctcgaggcgctgaaagaaaacgggggtgcccgcttggctgagtaccacgcgaaagcgacagaacacctgagcaccttgagcgaaaaagcgaaaccggcgctggaagatctacgccagggcttattgcctgttcttgagagctttaaagtcagttttctgtcagctctggaagaatatactaaaaagctgaatacccagggcacccccgtgacgcaggaattctgggacaacctggaaaaagaaaccgagggactgcgtcaggaaatgtccaaagatttagaagaggtgaaggccaaggttcagccatatctcgatgactttcagaaaaaatggcaggaagagatggaattatatcgtcaaaaggtggaaccgctgcgtgcggaactgcaagagggggcacgccaaaaactccatgagctccaagagaagctcagcccattaggcgaagaaatgcgcgatcgcgcccgtgcacatgttgatgcactccggactcatttggcgccgtattcggatgaacttcgccagcgtttggccgcacgtctcgaggcgctgaaagaaaacgggggtgcccgcttggctgagtaccacgcgaaagcgacagaacacctgagcaccttgagcgaaaaagcgaaaccggcgctggaagatctacgccagggcttattgcctgttcttgagagctttaaagtcagttttctgtcagctctggaagaatatactaaaaagctgaatacccagggcccgggaggccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 5 - protein, 820 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSFGGGGSHHHHHHHHHH

SEQ ID NO: 6 - protein, 507 aa, synthetic construct
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADGGGGGSHHHHHHHHHH

SEQ ID NO: 7 - protein, 488 aa, synthetic construct
MQVQLQESGPGLVKPSETLSLTCTVSGYSISSGYYWGWIRQPPGKGLEWIGSIYHSGSTYYNPSLKTRVTISVDTSKNQFSLKLSSVTAADTAVYYCARAVVGIVVVPAAGRRAFDIWGQGTMVTVSSGGGGSGGGGSGGGGSQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLVFGGGTKLTVLGPGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 975 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGPGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK